(19) United States
(12) Patent Application Publication
(10) Pub. No.: US 2004/0241202 A1
(43) Pub. Date: Dec. 2, 2004

(54) Biomaterials with bioactive coatings

(76) Inventors: Johanna Chluba, Chevigny-Saint-Sauveur (FR); Joella Ogier, Strasourg (FR); Jean-Claude Voegel, Valff (DE); Patrick Erbacher, Strasbourg (FR); Pierre Scharf, Molsheim (FR); Gero Decher, Kel Altemagne (DE)

Correspondence Address: NIXON & VANDERHYE, PC, 1100 N GLEBE ROAD, 8TH FLOOR, ARLINGTON, VA, 22201-4714, US

(21) Appl. No.: 10/475,278

(22) PCT Filed: Apr. 19, 2002
(86) PCT No.: PCT/FR02/01362

(30) Foreign Application Priority Data
Apr. 19, 2001 (FR) 01/05319

(51) Int. Cl. A61K 38/43

(52) U.S. Cl. 424/423; 424/94.1; 514/2

(57) ABSTRACT

The invention concerns biomaterials comprising a core coated with alternating layers of polyelectrolytes with opposite charges, characterised in that they include one or several biologically active molecules, with molecular weight less than about 10000 Da, said molecules being fixed to one or several layers of polyelectrolytes.

The invention relates to biomaterials comprising a core coated with alternating layers of polyelectrolytes which serve as anchoring means for fixing biologically active molecules.

The coating of materials using multilayers of polyelectrolytes allows the production of nanoarchitectures of interest for fixing biologically active molecules. These constructions have the advantage of being easy to produce and their manufacture can be adapted, practically, to any type of surface, whatever its shape, so long as it has at least a few charges.

Thus the fixing of antibodies under two bi-layers of polystyrene sulphonate/polyallylamine has been reported and a maintenance of the reactivity vis-a-vis antigens observed (see bibliographic reference (1) at the end of the description). Other work reports the fixing of enzymes (2). However, molecules that are of high molecular weight and charged are involved here.

Thus, while the prior art reports the integration of charged molecules, of high molecular weight, ranging for example from 12400 (cytochrome) to approximately 240000 (catalase), the inventors have shown that molecules of small size, if appropriate uncharged were able to be fixed or included in multilayers, while preserving their activity.

The work carried out has allowed it to be established that cells in culture were able to respond to small biologically active molecules, such as hormones, immobilized at the surface or incorporated in the multilayers of polyelectrolytes. It appeared that these molecules, when they are linked to the surface layer of the polyelectrolytes, retain the same biological activity as that observed with the free molecule. Moreover, the long term activity is maintained, when the molecule is embedded inside the multilayer architecture, and even under 50 layers, its short term activity depending on the depth of integration.

Therefore the aim of the invention is to provide novel biomaterials coated with multilayers of polyelectrolytes, with one or more biologically active molecules anchored to the layers of polyelectrolytes, the activity of which can be controlled and which, generally, can be stored in the dried form while preserving their activity.

The invention also relates to a process for obtaining such biomaterials, which is easy to put into practice.

A subject of the invention is also exploitation of these biomaterials in the medical field with the advantage, in particular, of being able to control their biological activity in a given application.

The work carried out has shown that the cells correspond in a specific manner to the small molecules.

The biomaterials of the invention comprise a core, for example an inert solid support or of gel type, coated with alternating layers of polyelectrolytes of opposite charges, and are characterized in that they comprise one or more biologically active molecules, of molecular weight less than approximately 10,000 Da, this or these molecules being fixed to one or more layers of polyelectrolytes.

Unexpectedly, as illustrated in the examples given hereafter, the cells in contact with these bioactive coatings can interact with the molecules situated at the surface and/or embedded in the multilayers.

The invention relates to biomaterials in which the molecules are fixed to the surface. As a variant, the biomaterials of the invention comprise the biologically active molecules anchored inside the multilayers and optionally also present at the surface.

Especially advantageous biomaterials contain these biologically active molecules at different depths in the multilayer architecture, which allows their controlled release over time and also their protection. It will be noted that the short-term response of the cells can then be modulated by varying the position of the biologically active molecules in the multilayers.

The invention thus relates to bioactive coatings in which the biologically active peptides have a molecular weight of less than 5,000 Da, even less than 2,000 Da, or even less than 1,000 Da.

In these different embodiments of the invention, the molecules can be identical or different.

More especially they are peptides or polypeptides, such as peptide hormones, antimicrobial peptides, anti-inflammatories, cytokines, growth factors, or also derivatives of amino acids allowing in particular grafting onto the polyelectrolytes. Particularly satisfactory results are obtained with molecules having a molecular weight of less of 200 Da such as O-methylserotonin.

The fixing of biologically active molecules is carried out by covalent coupling or, when the molecule is charged, by association.

The polyelectrolytes of the multilayers are chosen according to the biocompatibility desired for a given application, the half-life of the multilayers. These polyelectrolytes can be biodegradable or non-biodegradable. They are for example constituted by alternating layers of polylysine and polyglutamic acid.

Advantageously, these biomaterials can be stored for several weeks while retaining their activity. Advantageously their drying is carried out beforehand and with UV treatment.

The coatings according to the invention are advantageously obtained according to standard techniques used to produce multilayer architectures of polyelectrolytes.

The biomaterials of the invention are particularly valuable for applications in the medical field. They allow in particular the production of complex cell modulation systems at the surface of biomaterials.

The invention therefore relates to systems in which specific adhesion sequences are incorporated in order to target certain cell types, so as to induce the desired passages of signal transduction, and cell differentiation and simultaneously prevent the adhesion of undesired organisms with anti-microbial peptides or factors inhibiting the adhesion of micro-organisms.

The invention also relates to constructions with a biomaterial—extracellular matrix network after penetration of the cells in the multilayers by extension of cellular processes which can be used in localized therapies, for example in the form of patches, or generally in the form of implants, in order to release the therapeutically active products as hormones or others.

The bioactive coatings according to the invention are also of great interest for producing tissues in vitro, which are then implanted. It is thus possible to reconstruct a tissue on a biodegradable support, such as an inert solid material or a gel. If necessary, differentiation factors are added and the cells cultured.

Such modulatable bioactive surfaces constitute tools which allow the production of implant materials, in particular for orthopedic applications or for vascular uses, which can be adapted to the integration site.

Other characteristics and advantages of the invention are given in the examples which follow, which relate, by way of illustration, to the activity of 2 synthesis analogues of α-melanocortin or α-MSH, on a model constituted by murine melanoma cells.

α-MSH is a powerful stimulator of melanogenesis in the melanocytes of mammals and in melanoma cells. In the melanocytes or melanomas, α-MSH binds to a receptor specific to the cell surface, the melanocortin-1 receptor (MC1-R) and induces the activation of tyrosinase, a key enzyme for the formation of melanin, by stimulation of adenylate cyclase and protein kinase A. α-MSH is also a powerful anti-inflammatory agent in the brain and in the periphery and fixes to the melanocortin receptor of the macrophages or monocytes. α-MSH therefore constitutes a molecule of interest as a model for the study of the effects of signal transduction of cyclic AMP up to the final product, constituted in this case by melanin, a quantifiable pigment.

The 2 analogues are CP1 and CP2. They are derivatives of [Nle⁴, D-Phe⁷]-α-MSH (1-13) and [Nle⁴, D-Phe⁷]-α-MSH (4-10), obtained by grafting of the sequence HS—CH₂—CH₂—CO.

These derivatives of α-MSH are covalently bound to polylysine (PLL) in an architecture of alternating multilayers of polylysine and polyglutamic acid (PGA).

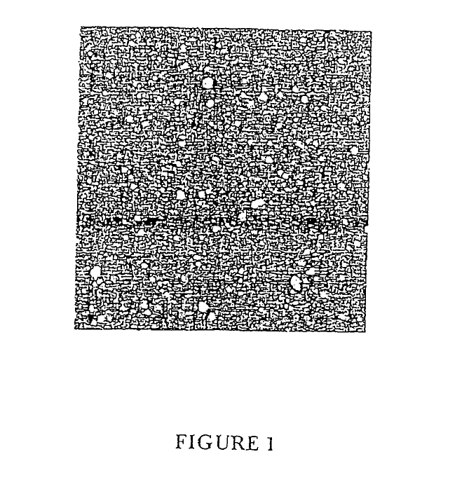

In these examples, reference is made to FIGS. 1 to 5, which represent, respectively,

FIG. 1: the AFM image of a multilayer of (PEI-PSS/PAH)₂ PGA/PLL-CP1-(PGA/PLL)₂ on a silica surface. This image was obtained using a Nanoscope III (Digital Instruments, Santa Barbara, Calif.) equipped with a cantilever made of silicon nitride (Model MLCT-AUHW Park Scientific, Sunnyvale, Calif.), having a spring constant of 0.03 N/m. The AFM is carried out in constant force contact mode and the surface scan is carried out with care applying the smallest force possible in order to avoid damaging the different samples.

A scan speed of between 2 and 4 Hz was applied, with an image pixel resolution of 512×512 pixels. The image size is 8×8 μm² and the z scale is 50 nm².

The rms of the z values over a surface of 25 μm² is typically 6 nm.

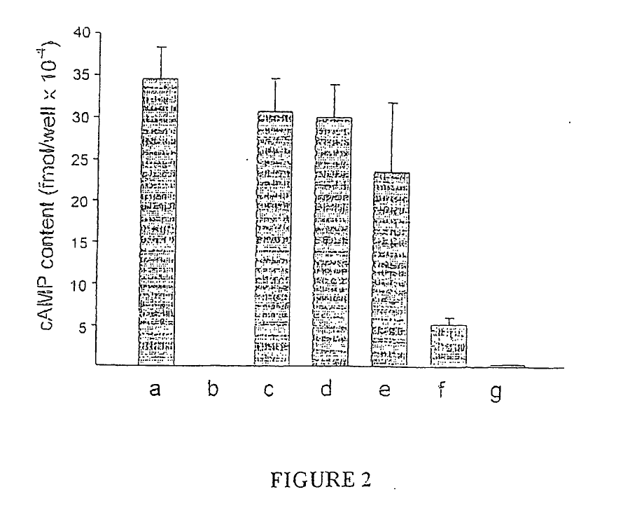

FIG. 2: the total cAMP content in the B16-F1 cells cultured on multilayer films containing the CP2 analogue of α-MSH. The cells are deposited on 24-well plates (Nunc) on multilayer films at a density of 40,000 cells/well. 2 hours after seeding, the cells are detached and the total cAMP content is measured by an immunoenzymatic assay.

The results are averages of three determinations of a representative experiment. This experiment is repeated twice with very similar results.

a: plastic+[NDP]-α-MSH; b: plastic; c: (PEI-PSS/PAH)₂ PGA/PLL-CP2; d: : (PEI-PSS/PAH)₂ PGA/PLL-CP2-(PGA/PLL)₅; e: : (PEI-PSS/PAH)₂ PGA/PLL-CP2)-(PGA/PLL)₁₀; f: (PEI-PSS/PAH)₂ (PGA/PLL-CP2)-(PGA/PLL)₂₅; g: (PEI-PSS/PAH)₂ (PGA/PLL)—(PGA/PLL)₂₅.

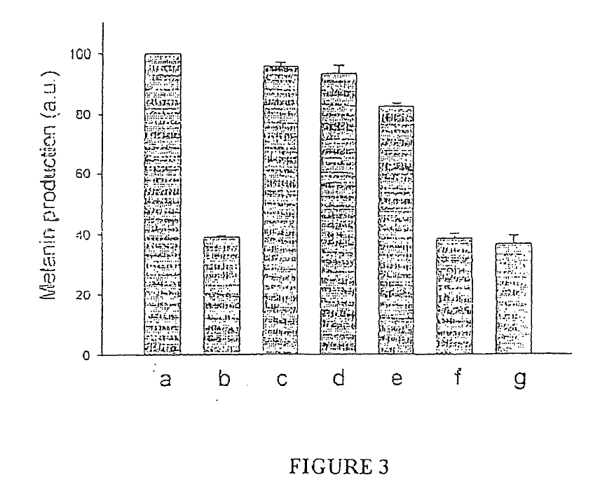

FIG. 3: the production of melanin in B16-F1 cells in contact with multilayer films containing an analogue of α-MSH. The cells are deposited on 24-well plates (Nunc) on multilayer films at a density of 25,000 cells/well. The melanin content (expressed in arbitrary units) was measured 4 days later. The coatings a to g are as indicated above compared with FIG. 2.

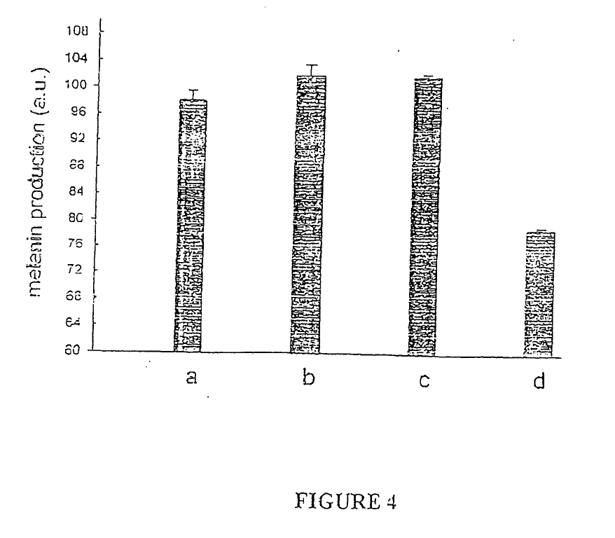

FIG. 4: the production of melanin in B16-F1 cells in contact with multilayers pre-washed with cell supernatant.

Before depositing the cells, multilayers are pre-washed for 24 hours with a supernatant of B16-F1 cells cultured for 2 hours in MEM without serum containing 0.2% of BSA.

The cells are deposited on 24-well plates, on the multilayers, at a density of 25,000 cells/well.

The melanin content (expressed in arbitrary units) was measured 4 days later.

a: (PEI-PSS/PAH)₂ PGA/PLL-CP1-(PGA/PLL)₅; b: (PEI-PSS/PAH)₂ PGA/PLL-CP1)-(PGA/PLL)₁₀; c: (PEI-PSS/PAH)₂ (PGA/PLL-CP1)-(PGA/PLL)₂₀; d: (PEI-PSS/PAH)₂ (PGA/PLL-CP1).

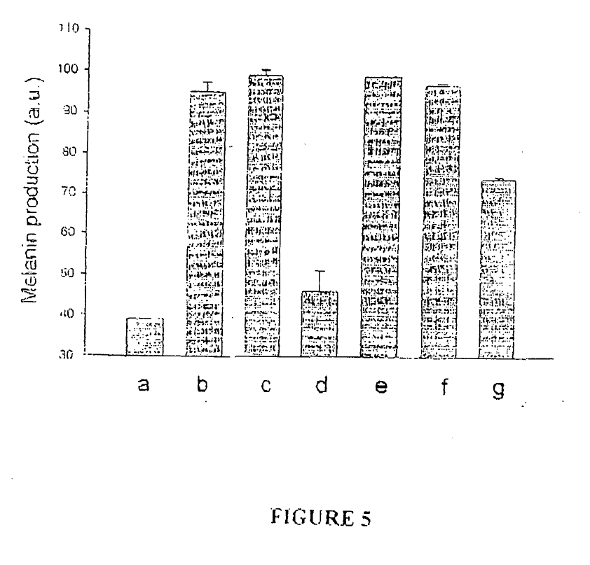

FIG. 5: the production of melanin in B16-F1 cells in contact with multilayer films containing an analogue of α-MSH coated with layers of (PSS/PAH). The cells are deposited on 24-well plates (Nunc) on multilayer films at a density of 25,000 cells/well. The melanin content (expressed in arbitrary units) was measured 4 days later.

a: plastic; b: PEI-PSS/PAH)₂ PGA/PLL-CP1-(PGA/PLL)₅; c: (PEI-PSS/PAH)₂ PGA/PLL-CP1)-(PGA/PLL)₁₀; d: (PEI-PSS/PAH)₂ (PGA/PLL)₁₀; e: (PEI-PSS/PAH)₂ (PGA/PLL-CP1); f: (PEI-PSS/PAH)₂-(PGA/PLL-CP1) (PSS/PAH)₄(PGA/PLL)₂; g: (PE-PSS/PAH)₂(PGA/PLL-CP1)-(PSS/PAH)₈(PGA/PLL)₂.

### SYNTHESIS OF PLL-PEPTIDE CONJUGATES

The peptide-PLL derivatives are prepared by conjugation using disulphide bridges.

The PLL is firstly functionalized with a thiol group, using succinimidyl 3-(2-pyridyldithio) propionate (SPDP, Sigma) (yield: 90%), then purified by carrying out several precipitations with isopropanol. The degree of modification is determined by reacting an aliquot with a excess of dithiothreitol and by monitoring the 2-thiopyridone at 343 nm.

The peptides containing thiol groups (CP1 etCP2) are added to a solution of PLL derivatized with dithiopyridine, and maintained under argon.

Measurements of the UV absorption of 2-thipyridone are carried out in order to monitor the reaction.

In order to reduce competition with the dimerization reaction of the peptide, 2 molar equivalents of peptide are added containing thiol, per dithiopyridine group. The conjugated products PLL-CP2 and PLL-CP1 are obtained, with yields of 0.43 and 0.76% respectively. The conjugates are purified by several purifications with isopropanol.

Cell Culture

B16-F1 cells are cultured in an Eagle medium (MEM) with Earle salts (Life Technologies) supplemented with 10% foetal calf serum inactivated by heat (30 min, 56° C.), 2 mM L-glutamine, 1% non-essential amino acids of mEM ((Life Technologies), 1.5% vitamin MEM solution (Gibco) and penicillin (50 units/ml)/streptomycin (50 μg/ml; Gibco).

Preparation of Multilayers

The multilayer films of polyelectrolytes are prepared in 24-well plates (Nunc), proceeding as follows:

Firstly, a precursor film of (PEI-PSS/PAH)₂ PGA is constructed on glass sheets, pre-treated with 10⁻²M SDS/0.12 N HCl, for 15 minutes at 100° C., by alternate immersion for 20 minutes in polyelectrolyte solutions (300 μl), at respective concentrations of 5 mg/ml in 1M NaCl for PEI, PSS/PAH and 1 mg/ml for PGA. Then PLL or PLL-CP1/2 is deposited on the layer of PGA over 12 hours. A PLL-CP1/2 concentration corresponding to 10⁻⁷ M of CP1 or CP2 is used.

After adsorption of the PLL-CP1/2, 300 μl of a solution of 1 mg/ml (1M NaCl) of non-modified PLL is added to the solution of PLL-CP1/2, in order to saturate the surface with the PLL.

Then n bilayers are constructed by the alternating deposition of the polyelectrolytes PLL and PGA (concentrations of 1 mg/ml in 1M NaCl). After the depositing of each layer of polyelectrolyte (PEI, PSS, PAH, PGA, PLL), the sheets are rinsed 3 times, for 5 minutes, with distilled water and dry air.

All the films are sterilized for 10 minutes by UV treatment (254 nm), stored dry, at ambient temperature and used within the week.

Before use, all the multilayers with non-cross-linked peptides, if present, are eliminated by washing, coating them with 2 ml of MEM without serum. The medium without serum is changed after 3, 6, 9, 24 hours, then the cells are seeded.

By optical spectrometry, with a waveguide, it is verified that the PLL-CP2 is adsorbed on the PGA end films and that it can be embedded in the multilayers of PGA/PLL.

The quantity of material deposited as well as the thickness of the film increases linearly with the number of bilayers in the presence or in the absence of PLL-CP2 in the molecule. The quantity adsorbed and the increase in the thickness are, respectively, of the order of 0.04 μg/cm and of 0.9 nm for the layers of PGA and PLL. These films are not dried during the construction. The uniformity, at cell level, of the deposited layer is confirmed by taking the AFM images in situ, as illustrated in FIG. 1.

The production of cyclic AMP (cAMP) and melanin was used to verify that the analogues of α-MSH retain their biological activity when they are covalently bound to PLL adsorbed or embedded in a multilayer.

The production of cAMP can be measured a few minutes after the activation, while the melanin content is measured after 4 days.

Determination of the Production of cAMP

The cells are detached from a cell culture flask with 0.02% of EDTA in 5×10⁻³ M of PBS, 0.2M of NaCl, 2.7×10⁻³M of KCl, counted, washed in MEM without serum and taken up in MEM containing 25 mM of HEPES and 0.2% of BSA. 40,0000 cells/well are seeded in 1 ml of the medium described above, using 24-well tissue-culture plates containing the glass sheets coated with the films.

After incubation for 2 hours, the total cAMP content is determined using a BIOTRAK EIA kit (Amersham Pharmacia Biotech), following the manufacturer's instructions.

The experiments are carried out in the absence of phosphodiesterase inhibitors.

The results are given in FIG. 2. The B16-F1 cells cultured on plastic and incubated with free [NDP]-α-MSH are used as positive controls, and the multilayers produced without hormone analogues as negative controls.

The CP2 bound to the external layer of PLL appears completely active.

It should be noted that the cells show a significant production of cAMP in response to CP2 even when the peptide, covalently bound to PLL, is embedded under 10 bilayers of PGA/PLL.

In fact, the total cAMP content remains practically constant when the CP2 is found just underneath 5 bilayers. 15% of the response is still observed when the CP2 is buried under 25 bilayers of PGA/PLL.

Quantification of Melanin

These tests allow the long-term response of the cells to PLL-CP2 included in the multilayer architecture to be determined. 25,000 cells are seeded in 24-well plates. The cell-culture medium is supplemented with 0.3 mM of L-tyrosine. The melanin content is quantified by determining the absorbence at 405 nm in a microplate reader after incubation for 4 days.

The results are given in FIG. 3. No significant difference is observed in the quantity of melanin produced when PLL-CP2 is at the surface or embedded under up to 25 bilayers of PGA/PLL. Similar results are obtained with CP1 instead of CP2.

In order to verify that the reduction in response of cells according to the position of CP 1 or CP2 in the film does not result from a degradation of the multilayers, the following tests are carried out.

Different multilayers containing PLL-CP1 with cell supernatants are pre-incubated, for 12 hours.

These supernatants are obtained from B16-F1 cells, cultured for 2 hours in MEM without serum, containing 0.2% of BSA, namely the same conditions as those used for the study of the production of cAMP. After several washings of the multilayers, the B16-F1 cells are cultured on these films and the melanin content is evaluated after 4 days.

The results are given in FIG. 4. A production of melanin when PLL-CP1 is embedded in the multilayers is clearly noted. On the other hand, when PLL-CP1 constitutes the external layer, the production of melanin decreases. Thus, it appears clear that the incubation with the supernatant cells does not lead to elimination of the hormone when it is included in the multilayers.

Study of the Process of Bringing the Cells into Contact with the Hormone

It appears that the cells need approximately 30 minutes to come into contact with the bioactive factors buried under 5 or even 10 bilayers of PGA/PLL.

Tests designed to avoid any degradation of the films were carried out.

Thus, non-biodegradable polyelectrolytes were incorporated in the multilayers. The results are illustrated in FIG. 5 and show that the insertion of 4 bilayers of polystyrene sulphonate (PSS)/polyallylamine (PAH) does not lead to a significant reduction in the production of melanin. It is observed that the insertion of 8 bilayers of PSS/PAH between PLL-CP1 and the external layer leads to a reduction of 25% in the production of melanin by the melanoma cells in contact with these surfaces. These results demonstrate the ability of the cells to come into contact with the active ingredient buried in the multilayers of polyelectrolytes even in the presence of non-degradable polyelectrolytes. The layers of PSS/PAH could thus form a barrier depending on the thickness of the layer and allow a modulation of the cell response.

### BIBLIOGRAPHIC REFERENCES

1. Caruso et al, Langmuir 13, 3427-3433, 1997.

2. Lvov et al, J. Am. Chem. Soc. 117,6117-6123, 1995

1. Biomaterial comprising a core coated with alternating layers of polyelectrolytes with opposite charges comprising one or more biologically active molecules, or these molecules being fixed to one or more layers of polyelectrolytes, characterized in that said biologically active molecule or molecules have a molecular weight less than approximately 10,000 Da.

2. Biomaterials according to claim 1, characterized in that the molecules are fixed at the surface.

3. Biomaterials according to claim 1, characterized in that the biologically active molecules are anchored inside the multilayers and optionally also present at surface.

4. Biomaterials according to claim 3, characterized in that the biologically active molecules are at different depths in the multilayer architecture.

5. Biomaterials according to claim 1 4, characterized in that the biologically active molecules are identical.

6. Biomaterials according to claim 1 4, characterized in that the biologically active molecules are different.

7. Biomaterials according to claim 1, characterized in that the biologically active molecules have a molecular weight of less than 5,000 Da, even less than 2,000 Da, or even less than 1000 Da.

8. Biomaterials according to claim 7, characterized in that the biologically active molecules are peptides, polypeptides, or derivatives of amino acids.

9. Use of the biomaterials according to claim 1 in the medical field.

10. Biomaterials according to claim 1, intended for use in localized therapy or for the reconstruction of tissues.

11. Use of a biomaterial according to claim 1 in the manufacture of a medicament intended to be used in localized therapy or for the reconstruction of tissues.
